(19) United States
(12) Patent Application Publication
(10) Pub. No.: US 2024/0201173 A1
(43) Pub. Date: Jun. 20, 2024

(54) Enamel Repair Visualization and Quantification

(71) Applicant: Colgate-Palmolive Company, New York, NY (US)

(72) Inventors: Qichao RUAN, Hillsborough, NJ (US); Stacey Lavender, Chesterfield, NJ (US)

(73) Assignee: Colgate-Palmolive Company, New York, NY (US)

(21) Appl. No.: 18/545,210

(22) Filed: Dec. 19, 2023

Related U.S. Application Data
(60) Provisional application No. 63/433,712, filed on Dec. 19, 2022.

(51) Int. Cl. G01N 33/50; G01N 33/483

(52) U.S. Cl. CPC G01N 33/5088; G01N 33/4833

(57) ABSTRACT

The present invention relates to methods for visualizing and quantifying the repair of tooth enamel, for example, for the evaluation of dentifrice compositions for the treatment or prevention of dental caries, early erosive damage, and enamel demineralization, and the promotion of enamel repair and remineralization.

### CROSS-REFERENCE TO RELATED APPLICATIONS

The present application claims priority to, and the benefit of, U.S. Provisional Application Ser. No. 63/433,712, filed on Dec. 19, 2022, the contents of which are hereby incorporated by reference in its entirety.

### FIELD

The present invention relates to methods for visualizing and quantifying the repair of tooth enamel, for example, for the evaluation of dentifrice compositions for the prevention or treatment of dental caries, early erosive damage, and enamel demineralization, and the promotion of enamel repair and remineralization.

### BACKGROUND

Dental plaque is a sticky biofilm or mass of bacteria that is commonly found between the teeth, along the gum line, and below the gum line margins. Dental plaque can give rise to dental caries and periodontal problems such as gingivitis and periodontitis. Dental caries tooth decay or tooth demineralization caused by acid produced from the bacterial degradation of fermentable sugar.

Enamel is the outermost layer of the mammalian tooth and the most mineralized tissue in the human body. It consists of over 95% by weight of minerals, and less than 5% by weight of water and organic materials. The primary mineral is elongated (carbonated) hydroxyapatite crystals that are densely packed and organized into an intricate interwoven structure. The high mineral content together with its organized structure provides enamel with significant resilience that protects the tooth from daily use such as chewing, biting, crunching, and grinding. Although enamel is a hard protector of teeth, it can be damaged by external chemical and physical insults. Unlike a broken bone that can be repaired by the body, the mature enamel is a non-living tissue and cannot regenerate itself after substantial mineral loss, which often occurs as dental caries or erosion.

Enamel erosion is one of the most common dental diseases and it is a growing problem. The development of enamel erosion involves a demineralization process characterized by acid dissolution of enamel crystals, not involving acids of bacterial origin. Since enamel erosion results in progressive and irreversible loss of mineralized tooth substance, the primary focus of erosion intervention is prevention and reduction, followed by management. Currently, the conventional method for preventing enamel erosion is to remineralize the softened enamel by providing essential ions, such as fluoride, calcium, and phosphate. It is well recognized that fluoride can promote the formation of fluoridate apatite on enamel and provide a better resistance to acid. Recent evidence indicates that materials containing calcium phosphate can also protect the teeth against erosion. Although these technologies have shown effects in protecting enamel, none of them could truly repair the enamel once it is damaged.

In addition, one of the hallmarks of early erosive tooth damage is “microdamage,” which refers to surface damage of the enamel visible only on a microscopic scale. Normal enamel surface has a fairly uniform smooth appearance. Acid damage causes a considerable roughening of the surface due to leaching of mineral ions (calcium and phosphate) from the surface structure (hydroxyapatite crystals). Physical damage can also occur, such as microscopic nicks and scratches in the surface of the enamel. Both of these types of microdamage can lead to localized areas of weakness and increased diffusion of acid and stains into the weakened enamel, as well as increased bacterial colonization.

Repairing the acid damaged demineralized enamel has been an important topic in the field of dentistry and material science. To evaluate the efficacy of enamel repair technologies, various characterization methods have been utilized to quantify or visualize the physical or morphological changes on enamel surfaces from different treatments. Typically, the enamel samples are examined by comparing the measurements before and after treatments. For example, the increase in the mechanical properties (such as micro-/nano-hardness and Young's modulus) have been recognized as evidence for an effective enamel repair treatment. Surface profilometry is another technique for measuring the surface roughness and recording step height that quantifies enamel loss regarding a non-treated area.

These methods have been well-established for quantifying the enamel repair efficacy. However, it is challenging to visualize the changes from treatment using only the mechanical or profilometry measurements. Several imaging techniques have been used for picturing the morphology on the enamel surface, such as the Light Microscopy, Confocal Laser Scanning Microscopy (CLSM), Scanning Probe Microscopes (SPM), and Scanning Electron Microscopy (SEM). Although these microscopic tools enable observation of microstructures on enamel surfaces, they have limited capability in quantifying the changes from the treatment in a typical before/after imaging procedure.

Additionally, the traditional method requires the separated measurements before and after treatments, that would increase the variability of samples and limit the accuracy for the assessments.

There remains a need for improved techniques for evaluating, visualizing, and quantifying enamel repair methods.

### BRIEF SUMMARY

Disclosed herein is a new methodology that is able to visualize the enamel changes from treatment while providing the capability for the quantification measurements from the same testing procedure and enamel sample.

The present methods provide for the use of a single block of enamel, divided into different sections for the different test conditions (e.g., negative control, positive control, and product under evaluation). The use of a single block having the side-by-side test conditions minimizes variation between samples, reduces the chances for error, and provides an improved means for visualizing and quantifying the results of the testing.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Schematic image for quantification and visualization of the enamel repair efficacy, as described in Example 2.

FIG. 2. Photomicroscopic images of enamel surface treated with water, sodium fluoride, or epsilon-polylysine/malic acid solutions, as described in Example 2. Scale bars are 50 microns in length.

FIG. 3. Color 3D images of enamel surface treated with water, sodium fluoride, or epsilon-polylysine/malic acid solutions, as described in Example 2.

FIG. 4. Scanning electron micrographs of enamel surface treated with water, sodium fluoride, or epsilon-polylysine/malic acid solutions, as described in Example 2. Scale bars are 2 microns in length.

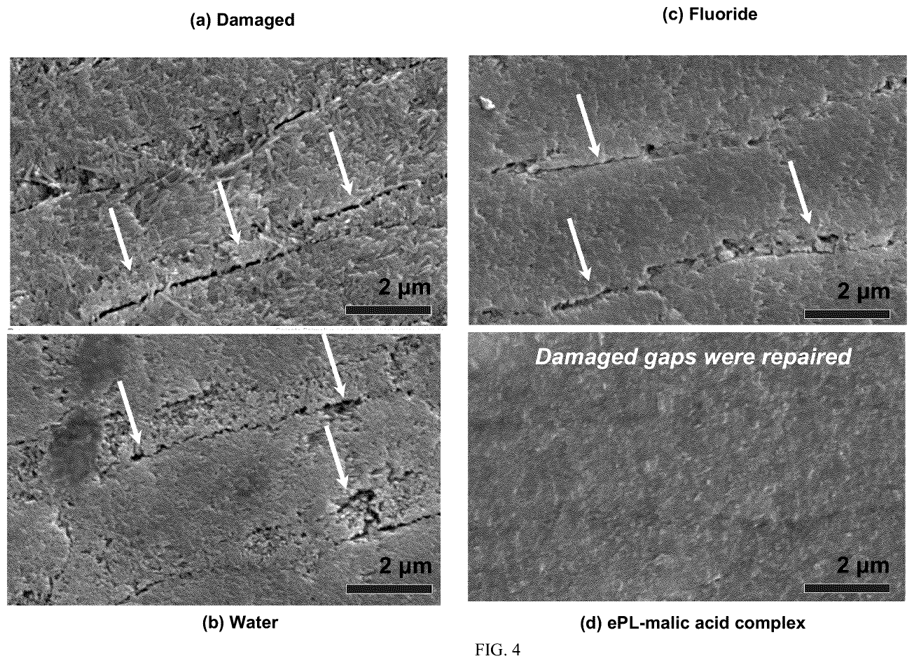

FIG. 5. Photomicroscopic images of enamel surface treated with sodium fluoride toothpaste slurry or water, as described in Example 3. Scale bars are 50 microns in length.

FIG. 6. Color 3D images of enamel surface treated with sodium fluoride toothpaste slurry or water, as described in Example 3.

FIG. 7. Scanning electron micrographs of enamel surface treated with sodium fluoride toothpaste slurry or water, as described in Example 3. Scale bar is 8 microns in length.

### DETAILED DESCRIPTION

The following description of the preferred embodiment(s) is merely exemplary in nature and is in no way intended to limit the disclosure, its application, or uses.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight of the entire composition. The amounts given are based on the active weight of the material.

The erosion of dental enamel results from acid dissolution of hydroxyapatite (HAP) crystals. The demineralization which results causes an uneven surface with a more random charge distribution compared to normal enamel. Normal remineralization of this uneven surface is achieved through a heterogeneous nucleation process controlled by gradient-based ion diffusion. This is a slow and very non-uniform process, and results in only limited natural repair at the surface.

Much current research in dentistry is directed to new means for the treatment of damaged enamel by remineralization, to reduce the roughness and unevenness of the surface.

The present disclosure provides a method (Method 1) for analyzing, visualizing, and/or quantifying, the repair of damaged tooth enamel resulting from the treatment of the damaged tooth enamel with a test composition, wherein the method comprises the steps of:
(a) providing a sample of human or animal tooth or enamel have a flat or substantially flat upper surface of enamel;
(b) covering a portion of the sample surface with an impermeable, removable coating (e.g., half of the sample surface), e.g., as a negative control;
(c) treating the uncoated portion of the sample surface with an agent to cause damage to the enamel of the uncoated sample surface;
(d) covering a portion of the damaged sample surface with an impermeable removable coating (e.g., half of the damaged sample surface), e.g., as a positive control;
(e) treating the remaining portion of uncoated, damaged sample surface with the test composition, e.g., as the test surface;
(f) removing the impermeable, removable coatings; and
(g) analyzing and comparing the differences between the three portions of the sample surface (the undamaged untreated negative control surface, the damaged untreated positive control surface, and the damaged treated test surface).

In further embodiments of Method 1, the present disclosure provides:
1.1 Method 1, wherein the sample of human or animal tooth or enamel is human tooth or enamel.
1.2 Method 1, wherein the sample of human or animal tooth or enamel is animal tooth or enamel (e.g., porcine or bovine tooth or enamel).
1.3 Method 1.1 or 1.2, wherein the sample is a whole or partial tooth, e.g., cut to remove the root and/or neck, such as to allow for mounting.
1.4 Method 1.1 or 1.2, wherein the sample is an enamel sample, e.g., a block of enamel cut from a tooth without dentin or other dental tissues.
1.5 Method 1.4, wherein the sample is a bovine enamel block, e.g., about 1-4 mm square and 1-3 mm in thickness.
1.6 Any of Methods 1.1-1.5, wherein the exposed enamel surface of the sample is ground and/or polished to provide a smooth and flat or substantially flat upper surface for testing.
1.7 Any of Methods 1.1-1.6, wherein the impermeable coating is acid-resistant.
1.8 Any of Methods 1.1-1.7, wherein the impermeable coating is a polymeric coating, e.g., a lacquer.
1.9 Method 1.8, wherein the impermeable coating is applied as a polymer suspended in a volatile organic solvent by brushing onto the enamel surface followed by air-drying, heat-drying, or vacuum-drying to remove the solvent.
1.10 Method 1.8 or 1.9, wherein the polymer is selected from nitrocellulose, cellulose acetate, cellulose acetate butyrate, a polyacrylate (e.g., polyacrylic acid, poly(methyl acrylate), poly(methyl methacrylate)), shellac, urushiol, polyacrylamides, and polyvinylpyrrolidone.
1.11 Method 1.9 or 1.10, wherein the volatile organic solvent is selected from acetone, methyl acetate, ethyl acetate, isopropyl acetate, butyl acetate, and the like.
1.12 Method 1 or any of 1.1-1.11, wherein the agent used to cause damage to the enamel surface is an acid (e.g., acetic acid, citric acid, hydrochloric acid, sulfuric acid, lactic acid).
1.13 Method 1.12, wherein the acid is applied as an aqueous solution, e.g., a 0.1-10 wt. % aqueous solution, or 0.1-5%, or 0.1-1% aqueous solution.
1.14 Method 1.13, wherein the acid is a 1 wt. % aqueous citric acid solution.
1.15 Any of Methods 1.1-1.14, wherein the agent used to cause damage to the enamel surface is applied by a dropper or brush.
1.16 Any of Methods 1.1-1.15, wherein step treatment of step (c) is carried out for 1-60 minutes, e.g., 1-40 minutes, or 1-30 minutes, or 1-20 minutes, or 1-15 minutes, or 1-10 minutes, or 1-5 minutes, or 1-3 minutes.
1.17 Any of Methods 1.1-1.15, wherein step treatment of step (c) is followed by rinsing, e.g., with water, to remove residual agent.
1.18 Any of Methods 1.1-1.17, wherein the impermeable coating of step (b) is the same as the impermeable coating of step (d).
1.19 Any of Methods 1.1-1.17, wherein the impermeable coating of step (b) is not the same as the impermeable coating of step (d).
1.20 Any of Methods 1.1-1.19, wherein the test composition is a dentifrice, e.g., a mouthwash, toothpaste, oral gel, or oral spray.
1.21 Any of Methods 1.1-1.19, wherein the test composition is an aqueous solution.
1.22 Any of Methods 1.1-1.21, wherein the test composition comprises an agent suspected to be effective in reducing or reversing enamel demineralization or enhancing or promoting enamel remineralization.
1.23 Any of Methods 1.1-1.22, wherein step (e) comprises treating the sample surface with the test composition for 1-60 minutes, e.g., 1-40 minutes, or 1-30 minutes, or 1-20 minutes, or 1-15 minutes, or 1-10 minutes, or 1-5 minutes, or 1-3 minutes, 1-2 minutes, or about 10 minutes, about 5 minutes, about 3 minutes, about 2 minutes, or about 1 minute, optionally followed by rinsing to remove the test composition.
1.24 Any of Methods 1.1-1.23, wherein the treatment of step (e) further comprises treating the sample of human or animal tooth or enamel, after application of the test composition, with a remineralization solution (e.g., an aqueous solution providing solubilized calcium and/or phosphate to provide ions for the growth of hydroxyapatite crystals in the enamel).
1.25 Method 1.24, wherein the remineralization solution comprises a water-soluble calcium salt and a water-soluble phosphate.
1.26 Method 1.24 or 1.25, wherein the remineralization treatment is carried out for at least 1 hour, or at least 2 hours, or at least 3 hours, or at least 6 hours, or at least 12 hours, or at least 24 hours.
1.27 Method 1.24 or 1.25, wherein the remineralization treatment is carried out for at least 1 day, e.g., 1 day, 2 days, 3 days, 4 days, 5 days, 6 days, or 7 days, optionally wherein the remineralization solution is freshly provided each day.
1.28 Any of Methods 1.1-1.27, wherein the impermeable coating is removed by treatment with an organic solvent in which the coating is soluble, e.g., acetone, toluene, ethyl acetate, dichloromethane, chloroform, carbon tetrachloride, acetonitrile, methanol, ethanol, or combinations thereof.
1.29 Method 1, or any of 1.1-1.28, wherein the analysis of step (g) comprises one or more of visual observation, photography, spectrographic analysis (e.g., ultraviolet/visible spectroscopy), light microscopy, confocal laser scanning microscopy (CLSM), scanning probe microscopy (SPM), and scanning electron microscopy (SEM).
1.30 Method 1.28, wherein the analysis of step (g) comprises 3D-image enhancement.
1.31 Method 1, or any of 1.1-1.30, wherein the test composition is a toothpaste, e.g., an aqueous slurry of a toothpaste (such as, a 1:2 w/w slurry of toothpaste to water).

The present disclosure also provides a method (Method 2) for evaluating a test composition for its ability to repair damaged tooth enamel or for determining the efficacy of a test composition in repairing damaged tooth enamel, wherein the method comprises the steps of:
(h) providing a sample of human or animal tooth or enamel have a flat or substantially flat upper surface of enamel;
(i) covering a portion of the sample surface with an impermeable, removable coating (e.g., half of the sample surface), e.g., as a negative control;
(j) treating the uncoated portion of the sample surface with an agent to cause damage to the enamel of the uncoated sample surface;
(k) covering a portion of the damaged sample surface with an impermeable removable coating (e.g., half of the damaged sample surface), e.g., as a positive control;
(l) treating the remaining portion of uncoated, damaged sample surface with the test composition, e.g., as the test surface;
(m) removing the impermeable, removable coatings; and
(n) analyzing and comparing the differences between the three portions of the sample surface (the undamaged untreated negative control surface, the damaged untreated positive control surface, and the damaged treated test surface).

The disclosure further provides the same embodiments 1.1-1.31 applied to Method 2.

The disclosure further provides an oral care composition comprising or consisting of a test composition evaluated or having its efficacy determined according to Method 2.

The invention of the present disclosure will be further described by way of the following non-limiting examples.

### EXAMPLES

### Example 1—Initial Evaluation of Epsilon-Polylysine Polymer Solution

Test solutions are prepared by mixing a solution of poly-epsilon-lysine (ePL; 25-35 lysine residues, about 3200-4600 Da) with malic acid solution in appropriate ratios to provide the following formula. The solution is adjusted to about pH 6.0 using 1M aqueous NaOH:

| Sample | A |
|---|---|
| ePL (mg/mL) | 10 |
| Malic acid (mg/mL) | 0.06 |
| PL/ MA weight ratio | 1:0.006 |
| Amino/carboxy | 1:0.01 |
| molar ratio | |

Bovine enamel blocks are obtained from sound bovine incisors without defects. The labial surface of the bovine teeth is cut to provide enamel specimens of about 3×3×2 mm in size. The enamel layer is about 1 mm thick and the dentin left in the specimen is about 1 mm thick.

Etched enamel samples are prepared by treatment with 1% aqueous citric acid (pH 3.6) for 10 minutes, followed by rinsing with deionized water. Three bovine enamel blocks are prepared, one for the test solution, one for a positive control solution, and one for a negative control solution.

Etched bovine enamel samples are treated with 40 microliters of the polyelectrolyte solution A, with sodium fluoride solution, or with water, for ten minutes at room temperature. The samples are then rinsed with deionized water and then they are immersed in 50 mL of Remineralization Solution (0.2205 g/L CaCl₂·2H₂O, 0.1225 g/L KH₂PO₄, 9.6915 g/L KCl and 4.766 g/L HEPEs buffer, pH adjusted to 7 with NaOH) overnight at 37° C. The enamel samples are then rinsed with deionized water for 50 seconds and then air dried.

Knoop Hardness (KHN) is a measure of hardness of a material calculated by measuring the indentation produced by a diamond tip that is pressed onto the surface of a sample. Using a calibrated microscope, the area of the impression can be determined, and from the area of the indentation (D) in square millimeters, and the applied load (F) in kilograms force, the Knoop Hardness is calculated as: KHN=14.229(F/D²). Knoop hardness has units of kg_{f}/mm².

KHN is determined using Knoop microhardness tester. A baseline measurement is taken on a sound enamel sample without treatment (KHN₀), on an acid-etched enamel sample (KHNₑ), and on an acid-etched enamel sample after treatment (KHNₜ). Each KHN measurement is the average of 5 indentations made on the same specimen at a force of 0.05 kg for 15 seconds. A value for Percentage of Hardness Repair (% HR) is calculated according to the following formula:

$$\% H R = ( K H {N}_{t} - K H {N}_{e} ) / ( KH {N}_{0} - K H {N}_{e} ) \times 100$$

Distilled water (pH 6-7) and 1450 ppm sodium fluoride in distilled water solution (0.15% w/v, pH 7.3) are used as controls. The results of the bovine enamel tests are shown in the table below:

| Sample | A | NaF Control | H₂O Control |
|---|---|---|---|
| % HR | 84.9 | 41.0 | 22.4 |

The results show that surface hardness was slightly increased after 1 day of treatment with water, while sodium fluoride treatment resulted in a more significant improvement, but did not reach half of original hardness levels. Treatment with the Sample A, having 10 mg/mL ePL with malic acid results in a substantial and statistically significant increase in repair compared to the controls. 10 mg/mL ePL with malic acid restores the enamel to more than 80% of the original hardness level.

These results establish the effectiveness of the test treatment using standard testing conditions.

### Example 2—Improved Method for Enamel Repair Evaluation-Solution Samples

An improved method of evaluating enamel repair is developed in order to more quickly and effectively show improvement. The method is tested on the same composition as described in Example 1. The method relies on the use of a single bovine enable block instead of three separate blocks.

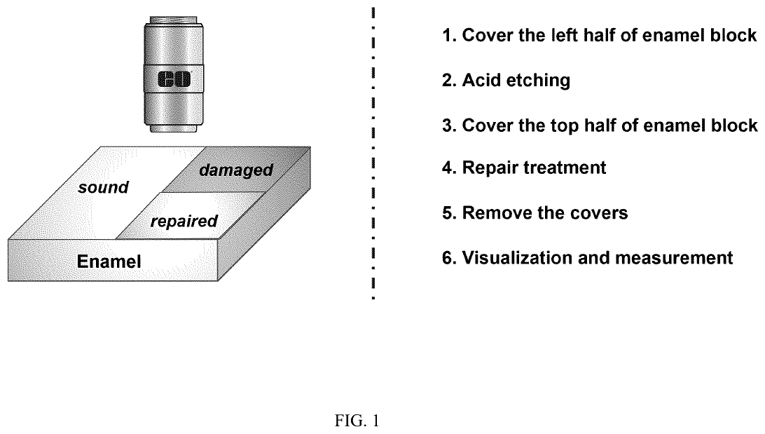

As shown in FIG. 1, bovine enamel blocks are divided into three sections, termed “sound” (i.e., undamaged), “damaged” and “repaired.” Polished bovine enamel blocks having a hardness of at least 300 KHN are selected and cut into square blocks of about 3×3 mm and about 2 mm thickness. The left half of the block (“sound”) is covered using an acid-resistant nail polish, which is then allowed to air dry. The right half of the block (“damaged” and “repaired”) is then treated with 1% citric acid (pH 3.6) for ten minutes to etch the surface. After the treatment, the surface of the block is rinsed with deionized water. The top right corner of the block (“damaged”) is then covered with the same acid-resistant nail polish and air dried. The test formulation is then applied to the bottom right corner of the block (“repaired”), for example, for a ten-minute treatment, followed by rinsing with deionized water. Following the treatment, the enamel blocks are immersed in a Remineralization Solution (0.2205 g/L CaCl₂·2H₂O, 0.1225 g/L KH₂PO₄, 9.6915 g/L KCl and 4.766 g/L HEPEs buffer, pH adjusted to 7 with NaOH) overnight at 37° C., and the process repeated for 2-4 days (the blocks immersed daily in fresh remineralization solution). Following this, the blocks are removed, rinsed with deionized water and air dried. The nail polish covers are then removed using acetone solvent, and the surface of the enamel blocks is visualized, for example, by light microscopy, confocal laser scanning microscopy, scanning probe microscopy, or scanning electron microscopy.

A Keyence microscope is used to capture the surface morphology and to measure the step height of the treated samples. A Phenom ProX Desktop scanning electron microscope is used to observe the repaired enamel crystals. Bovine enable blocks are treated with either water, the sodium fluoride solution, or the 10 mg/mL ePL+0.06 mg/mL malic acid solution (Solution A above).

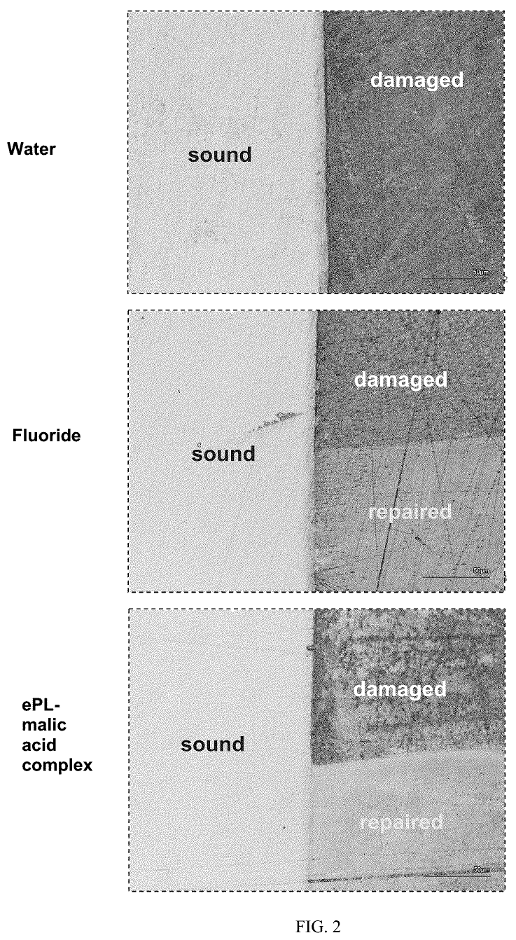

As shown in FIG. 2, the polished “sound” portion of the enamel exhibits a smooth and bright appearance under the microscope. The acid etching procedure causes a dissolution of enamel crystals resulting in a rough and dark surface under the microscope, as seen in the “damaged” portions of the enamel. The remineralization process, with the negative control water treatment, did not change the surface morphology (i.e., the “damaged” and the “repaired” surfaces appear identical). In contrast, a clear interface between “damaged” and “repaired” sides is observed with the fluoride treated sample. The sample treated with the ePL-malic acid complex, however, shows that the “repaired” side is substantially smoother and brighter compared to the other samples, indicating a superior repair efficacy compared to other treatments.

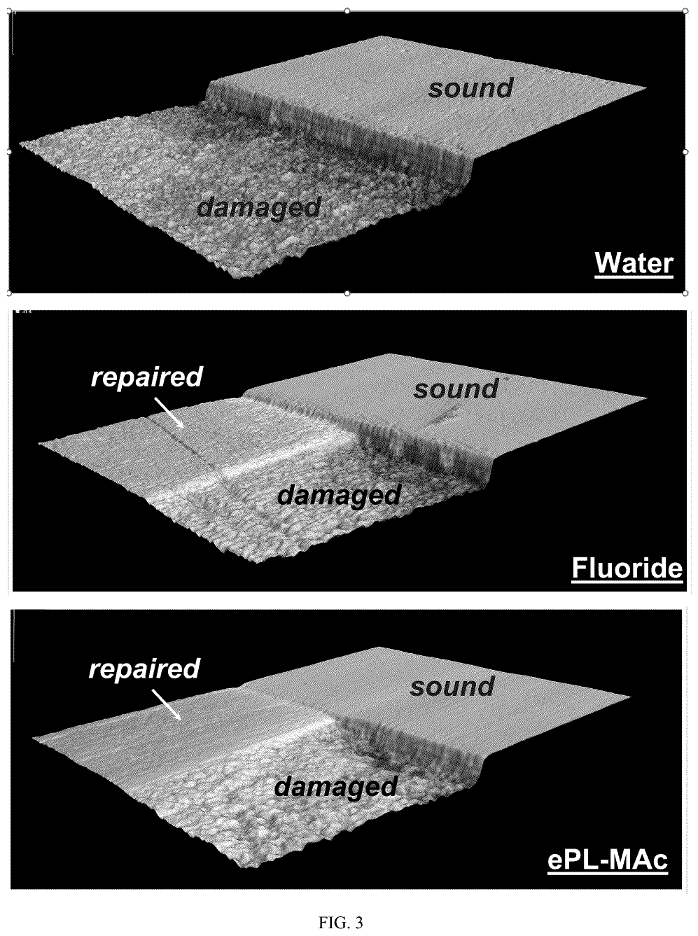

FIG. 3 shows 3-D Keyence photomicrographs color-enhanced to show the surface elevation of the enamel blocks. From these images, the step between the “sound” and “damaged” enamel portions is clearly observed, indicating a loss of minerals from the acid etching challenge. Water and fluoride treatments do not restore the enamel to the original height, but impressively, the treatment using ePL-malic acid complex restores the enamel level substantially to the height of the “sound” enamel side. Based on these images, the percentage of repair was calculated based on the change in the step height, as shown in the table below:

| Sample | E | NaF | H₂O |
|---|---|---|---|
| % Repair (Step height) | 87.0 | 65.3 | 30.9 |

As shown in the table, nearly 90% of recovery in step height is achieved by applying the ePL-malic acid complex on the enamel surface, which is significantly better than the results for water and fluoride treated samples.

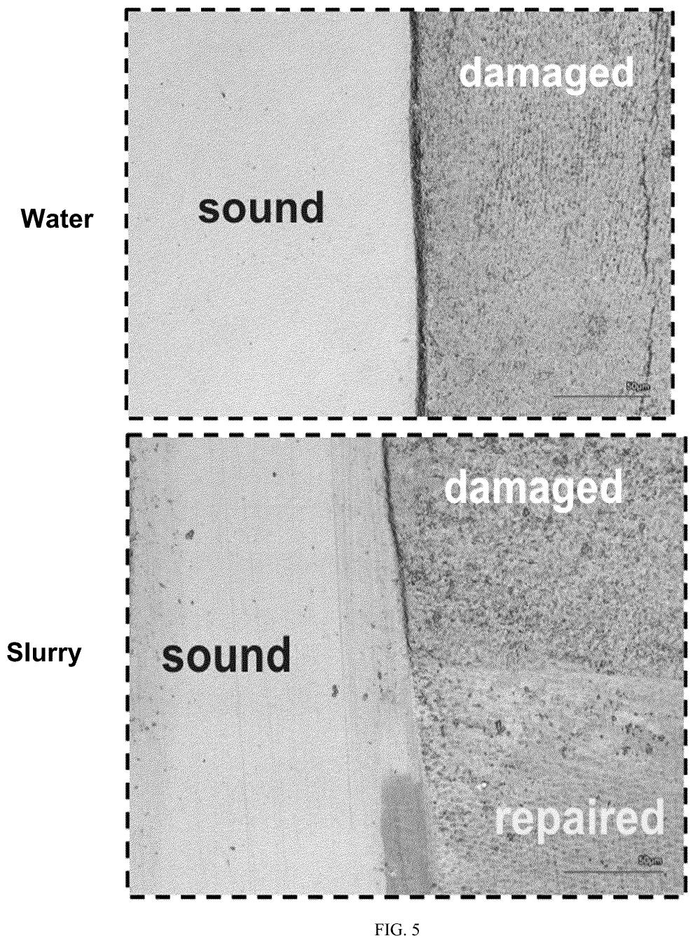

Finally, scanning electron microscopy is used to provide a closer view at the enamel surface morphologies. As shown in FIG. 5, the results further demonstrate the effectiveness of ePL-malic acid complex enamel repair. Acid etching is shown to cause gaps or cracks to form in the surface of the enamel due to the dissolution of enamel crystals (see arrows in the FIG. 5, panel (a)). The remineralization process alone is not able to repair these gaps effectively, due to the limited crystallization on enamel surface (panel (b)). The damaged enamel is found to undergo some healing after being treated with fluoride, however, the gaps still could be seen on the enamel surface (panel (c)). In contrast, treatment with the ePL-malic acid complex completely repairs all gaps observed in the enamel, restoring a smooth surface (FIG. 5, panel (d)).

These results demonstrate the highly efficient methodology of the present invention.

### Example 2—Improved Method for Enamel Repair Evaluation-Toothpaste Samples

The procedure described in Example 2 is also carried out using a slurry of a sodium-fluoride based toothpaste, rather than a simple solution. This test is carried out to demonstrate that the method is practical and effective for a commercial development product. The formula of this toothpaste is shown in the following Table:

| Component | Weight % |
|---|---|
| Sodium Fluoride | 0.32% |
| Zinc Citrate | 0.5% |
| Zinc Oxide | 1% |
| Arginine | 1.5% |
| Humectants | 35% |
| Abrasives | 15% |
| Thickeners | 15% |
| Polymers | 1.4% |
| Surfactants (e.g., anionic and/or | 5.5% |
| zwitterionic surfactants) | |
| Anticalculus agents (alkali phosphates) | 0.5% |
| Preservatives, buffers, flavors, | 3.25% |
| sweeteners, and colors | |
| Water | Q.S. |

The procedure described in Example 1 is modified as follows. First, the citric acid etching step is carried out for 20 minutes instead of 10 minutes. Second, a toothpaste slurry is prepared by combining one part of toothpaste (by weight) with 2 parts of deionized water (by weight) and the two are mixed thoroughly for 30 minutes. The enamel block is then soaked in this toothpaste slurry, using 2 mL of slurry for one block. After ten minutes at room temperature, the block is rinsed with deionized water, and the remainder of the Example 2 procedure is carried out.

As shown in FIG. 5, the polished “sound” portion of the enamel exhibits a smooth and bright appearance under the microscope, while the acid-etched “damaged” portion shows a rough and dark surface. The remineralization process, with the negative control water treatment, did not change the surface morphology (i.e., the “damaged” and the “repaired” surfaces appear identical). In contrast, a clear interface between the “damaged” and “repaired” sides is observed with the toothpaste slurry-treated sample.

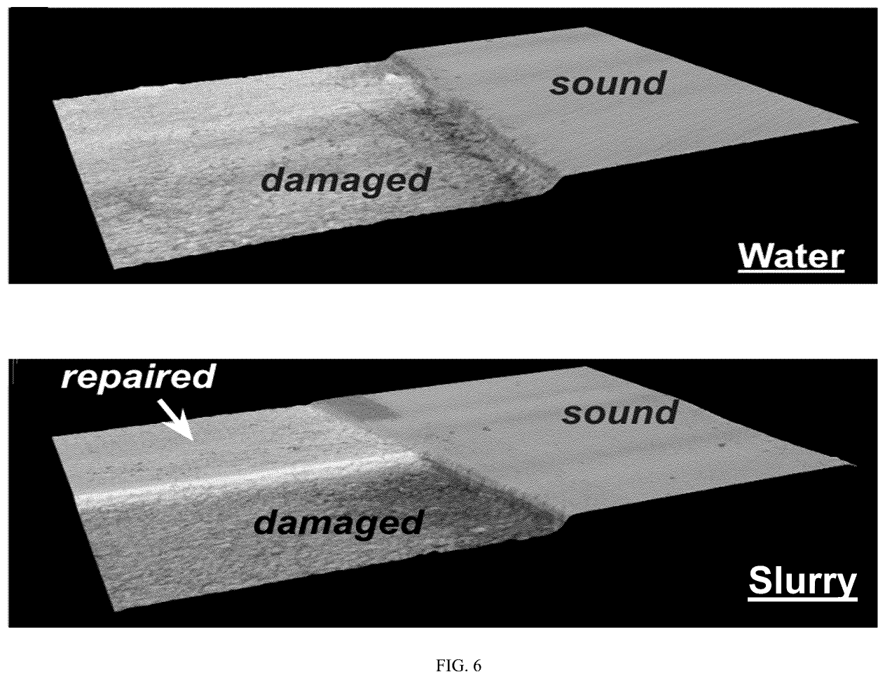

FIG. 6 shows 3-D Keyence photomicrographs color-enhanced to show the surface elevation of the enamel blocks. From these images, the step between the “sound” and “damaged” enamel portions is clearly observed, indicating a loss of minerals from the acid etching challenge. Water treatment does not provide any significant restoration of the enamel, but the treatment using the fluoride-based toothpaste does increase the height of the “sound” enamel side. Based on these images, the percentage of repair was calculated based on the change in the step height, as shown in the table below:

| Sample | Toothpaste slurry | Water |
|---|---|---|
| % Repair (Step height) | 55.6 | 24.1 |

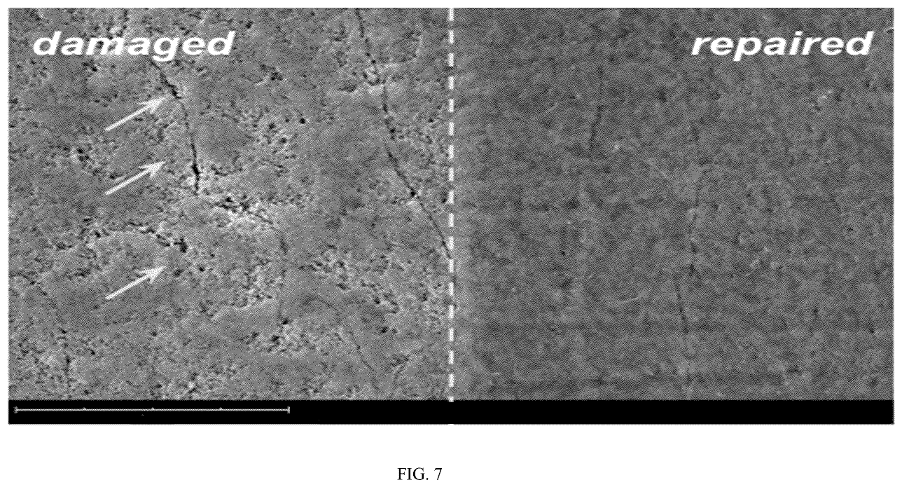

Finally, scanning electron microscopy is used to provide a closer view at the enamel surface morphologies. As shown in FIG. 7, the results further demonstrate the repair of enamel etching by the toothpaste slurry. Acid etching is shown to cause gaps or cracks to form in the surface of the enamel due to the dissolution of enamel crystals (see arrows in the FIG. 7, left panel). The remineralization process alone is not able to repair these gaps effectively, due to the limited crystallization on enamel surface (data not shown) Treatment with the fluoride-based toothpaste results in significant repairs of the gaps observed in the enamel, improving the smoothness of the (FIG. 7, right panel).

These results further demonstrate the highly efficient methodology of the present invention for evaluating enamel repair.

While the present invention has been described with reference to embodiments, it will be understood by those skilled in the art that various modifications and variations may be made therein without departing from the scope of the present invention as defined by the appended claims.

1. A method for analyzing, visualizing, and/or quantifying, the repair of damaged tooth enamel resulting from the treatment of the damaged tooth enamel with a test composition, wherein the method comprises the steps of:
(a) providing a sample of human or animal tooth or enamel have a flat or substantially flat upper surface of enamel;
(b) covering a portion of the sample surface with an impermeable, removable coating (e.g., half of the sample surface), e.g., as a negative control;
(c) treating the uncoated portion of the sample surface with an agent to cause damage to the enamel of the uncoated sample surface;
(d) covering a portion of the damaged sample surface with an impermeable removable coating (e.g., half of the damaged sample surface), e.g., as a positive control;
(e) treating the remaining portion of uncoated, damaged sample surface with the test composition, e.g., as the test surface;
(f) removing the impermeable, removable coatings; and
(g) analyzing and comparing the differences between the three portions of the sample surface (the undamaged untreated negative control surface, the damaged untreated positive control surface, and the damaged treated test surface).

2. The method of claim 1, wherein the sample of human or animal tooth or enamel is human tooth or enamel.

3. The method of claim 2, wherein the sample is a bovine enamel block, e.g., about 1-4 mm square and 1-3 mm in thickness.

4. The method of claim 1, wherein the impermeable coating is acid-resistant.

5. The method of claim 1, wherein the impermeable coating is a polymeric coating, e.g., a lacquer.

6. The method of claim 1, wherein the agent used to cause damage to the enamel surface is an acid (e.g., acetic acid, citric acid, hydrochloric acid, sulfuric acid, lactic acid).

7. The method of claim 6, wherein the acid is a 1 wt. % aqueous citric acid solution.

8. The method of claim 1, wherein the test composition is a dentifrice, e.g., a mouthwash, toothpaste, oral gel, or oral spray.

9. The method of claim 8, wherein the test composition is a toothpaste, e.g., an aqueous slurry of a toothpaste (such as, a 1:2 w/w slurry of toothpaste to water).

10. The method of claim 1, wherein the test composition is an aqueous solution.

11. The method of claim 1, wherein the treatment of step (e) further comprises treating the sample of human or animal tooth or enamel, after application of the test composition, with a remineralization solution (e.g., an aqueous solution providing solubilized calcium and/or phosphate to provide ions for the growth of hydroxyapatite crystals in the enamel).

12. The method of claim 1, wherein the analysis of step (g) comprises one or more of visual observation, photography, spectrographic analysis (e.g., ultraviolet/visible spectroscopy), light microscopy, confocal laser scanning microscopy (CLSM), scanning probe microscopy (SPM), and scanning electron microscopy (SEM).

13. The method of claim 1, wherein the analysis of step (g) comprises 3D-image enhancement.
